(19) United States
(12) Patent Application Publication
(10) Pub. No.: US 2024/0376534 A1
(43) Pub. Date: Nov. 14, 2024

(54) MASTER MIX COMPOSITIONS, KITS, AND METHODS

(71) Applicant: LIFE TECHNOLOGIES CORPORATION, Carlsbad, CA (US)

(72) Inventors: Kalpith RAMAMOORTHI, San Jose, CA (US); Noah ELDER, Lafayette, CA (US)

(21) Appl. No.: 18/689,573

(22) PCT Filed: Sep. 9, 2022
(86) PCT No.: PCT/US2022/076204

§ 371 Date: Mar. 6, 2024

Related U.S. Application Data
(60) Provisional application No. 63/242,835, filed on Sep. 10, 2021.

(51) Int. Cl. C12Q 1/686

(52) U.S. Cl. CPC C12Q 1/686

(57) ABSTRACT

Disclosed are master mix compositions, kits, and related methods for use in amplifying a target nucleic acid. A master mix composition may be formulated to enable effective reaction mixture loading onto a dPCR plate, provide high proportion of valid/readable partitions, enable identification and distinguishing between amplified and unamplified partitions, enable detection of low abundance targets amidst high background levels of non-target nucleic acids and/or amidst inhibiting agents, and/or enable effective detection of low frequency mutant alleles.

### CROSS-REFERENCE TO RELATED APPLICATIONS

This PCT application claims the benefit of U.S. provisional application No. 63/242,835, filed on Sep. 10, 2021. To the extent permitted in applicable jurisdictions, the entire contents of this application are incorporated herein by reference.

### BACKGROUND

This disclosure relates to compositions, kits, and methods for the amplification and/or detection of nucleic acids.

For many medical, diagnostic, and forensic applications, amplification of a particular nucleic acid sequence is essential to allow its detection in, or isolation from, a sample in which it is present in very low amounts.

Polymerase chain reaction (PCR) is an in vitro method for the enzymatic synthesis of specific DNA sequences using two oligonucleotide primers that hybridize to opposite strands and flank the region of interest in target DNA. A repetitive series of reaction steps involving template denaturation, primer annealing, and the extension of the annealed primers by DNA polymerase results in the exponential accumulation of a specific fragment whose termini are defined by the primers. PCR can selectively enrich a specific DNA sequence by several orders of magnitude.

PCR “master mixes” improve the efficiency of amplification reactions. These master mixes contain a combination of reagents common to most PCR reactions such as a buffer, a salt such as MgCl₂, deoxynucleotide triphosphates (dNTPs), and a DNA polymerase. When performing PCR, each reaction volume includes the master mix and a specific target nucleic acid and primer pair. Typically, master mixes are manufactured and distributed as concentrated solutions or lyophilized powders which are subsequently diluted or dissolved when final reactions are assembled.

Digital polymerase chain reaction (dPCR) is a specific type of PCR that can be used to directly quantify target nucleic acids. In dPCR, the reaction mixture is partitioned into many small reaction volumes (i.e., partitions) so that the target nucleic acid is in some, but not all, of the reaction volumes/partitions. The reaction volumes are subjected to thermal cycling, and the proportion of “positive” partitions that generate a signal (usually a fluorescence signal) indicative of the presence of the target is determined. Quantitation is based on application of Poisson statistics, using the number of negative/non-reactive reaction volumes and assuming a Poisson distribution to establish the number of initial copies that were distributed across all the reaction volumes.

Problems with dPCR arise when the assumption of a Poisson distribution is not met. For example, the dPCR process assumes substantially even distribution of the reaction mixture and random partitioning of the target among the partitions of a dPCR reaction. When these assumptions are not met, the result lowers the reliability and accuracy of the dPCR results. For example, artifacts associated with the microfluidic loading of the reaction mixture into the multiple partitions can introduce unwanted and/or unknown variables that can negatively impact the accuracy, precision, and/or reliability of the results.

There is an ongoing need for improved master mix compositions, and particularly there is an ongoing need for master mix compositions that provide one or more benefits to dPCR applications.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification, wherein:

FIGS. 1A and 1B illustrate results of testing a master mix composition as disclosed herein to determine whether a low abundance target (0.5 copies/μL) could be detected with 95% confidence relative to the NTC (no template control) using dPCR, the results showing that measured values of the low abundance target were different to a statistically significant degree from the NTC

FIGS. 2A and 2B illustrate results of testing a master mix composition as disclosed herein to determine whether a rare mutant allele could be effectively detected in the presence of an inhibitor using dPCR, the results showing that 0.1% mutant alleles were effectively detected in the presence of high levels of background DNA and even in the presence of 50 μM/reaction hematin;

FIGS. 3A and 3B illustrate average of percent rain for various assay kits utilizing a master mix as disclosed herein, showing that percent rain was minimized for the dPCR plate;

FIGS. 4A and 4B illustrate average resolution for various assay kits utilizing a master mix as disclosed herein, showing that effective resolution was consistently provided; and

FIG. 5 illustrates results of testing different concentrations of master mix surfactant in dPCR, showing that sufficient levels of surfactant are beneficial to enable effective distinguishing between positive and negative partitions;

FIG. 6 compares images showing positive partitions of a dPCR plate where the master mix included sufficient surfactant and a dPCR plate where the master mix included insufficient surfactant; and

FIG. 7 compares images showing the passive reference signal for a dPCR plate with master mix that did not include a crowding agent to a dPCR plate with master mix that did include a crowding agent, showing that inclusion of sufficient crowding agent in the master mix evens plate loading and reduces reagent loading artifacts.

### DETAILED DESCRIPTION

### Overview of Master Mix Compositions

Master mix compositions described herein enable effective amplification of target nucleic acids in an amplification reaction. Many of the examples described herein are provided in the context of a dPCR application. The skilled person will understand, however, that the disclosed master mix compositions may be utilized in other nucleic acid amplification processes such as reverse transcription PCR (RT-PCR) and/or quantitative/real-time PCR (qPCR).

Although not limited to dPCR, the master mix compositions described herein have been found to be particularly effective for dPCR applications. For example, the master mix compositions described herein enable effective reaction mixture loading onto a dPCR plate, provide high proportion of valid/readable partitions, enable identification and distinguishing between amplified and unamplified partitions, enable detection of low abundance targets amidst high background levels of non-target nucleic acids and/or amidst inhibiting agents, and/or enable effective detection of low frequency mutant alleles.

Exemplary master mix component and component amounts are described below. Typically, a master mix is provided as a concentrated stock solution intended for mixing with other components of the reaction (e.g., the sample itself, other optional buffers, nuclease free water, etc.) to form the full reaction mixture. The component amounts disclosed below can therefore be understood to refer to a concentrated stock solution intended for dilution in the resulting reaction mixture. The stock solution may be formulated as a 2×, 3×, 4×, 5×, 6×, 7×, 8×, 9×, or 10× solution, for example.

In one embodiment, a master mix composition comprises a DNA polymerase, a crowding agent, and a surfactant. The surfactant is included at a concentration of at least 0.05% (w/v) or greater. In some embodiments, the surfactant is included at a concentration of at least 0.055%, 0.06%, 0.065%, 0.07%, 0.075%, 0.08%, 0.085%, 0.09%, 0.095%, 0.1%, 0.105%, or 0.11% (w/v) or greater. For example, the surfactant may be provided at a concentration between 0.05% and 0.2%, or between 0.06% and 0.18%, or between 0.07% and 0.16%, or between 0.07% and 0.15%, or between 0.08% and 0.125%, between 0.09% and 0.12%, or within a range having endpoints defined by any two of the foregoing values.

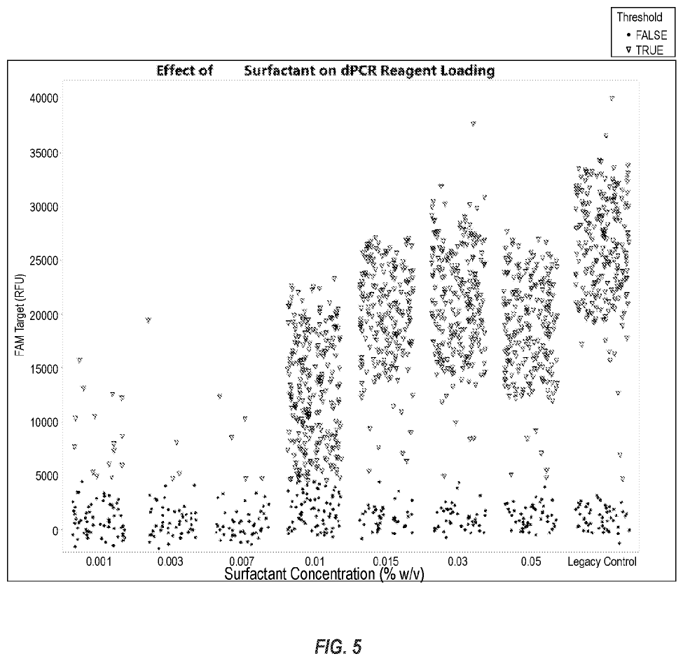

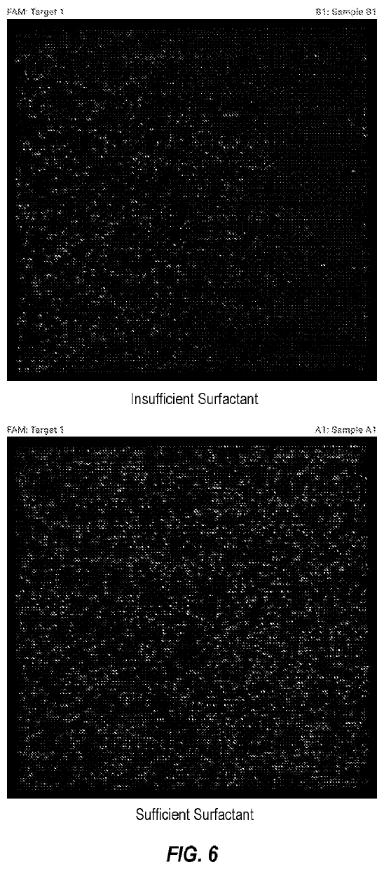

As described in more detail below, providing a surfactant at these concentrations was found to enable effective reaction mixture loading onto dPCR plates and to beneficially enable effective identification of amplified and unamplified partitions of the dPCR plate. In contrast, insufficient levels of surfactant led to inability to accurately identify partitions that included the target sequence. In particular, insufficient surfactant levels were associated with an inaccurate number of positive partitions and/or with inability to effectively distinguish between positive and negative partitions because many measured signals were close to the fluorescence threshold. Ideally, the measured fluorescence signal from a positive partition (one that includes the target nucleic acid) is well above the selected threshold while negative partitions (those without the target nucleic acid) are below the threshold. Using surfactant at the foregoing concentrations beneficially resulted in grouping of positive and negative partitions rather than forming a continuum or gradient of different signals with many close to the threshold (see FIG. 5 and discussion in Example 8).

In some embodiments, the surfactant includes one or more nonionic surfactant. Examples include polysorbate 20 (TWEEN 20), polysorbate 80 (TWEEN 80), 2-[4-(2,4,4-trimethylpentan-2-yl)phenoxy]ethanol (Triton X-100), an alcohol ethoxylate (e.g., Ecosurf EH-9, TERGITOL 15-S-9), nonyl phenoxypolyethoxylethanol (NP-40), 3-((3-cholamidopropyl) dimethylammonio)-1-propanesulfonate (CHAPS), 3-[(3-cholamidopropyl)dimethylammonio]-2-hydroxy-1-propanesulfonate (CHAPSO), polyoxyethylene (23) lauryl ether (Brij-35), polyoxyethylene (20) cetyl ether (Brij-58), polyoxyethylene (20) stearyl ether (Brij-78), or polyoxyethylene (20) oleyl ether (Brij-98).

In some embodiments, the surfactant additionally or alternatively includes a cationic or zwitterionic surfactant. Examples include benzalkonium chloride (BZK), didodecyldimethylammonium bromide (DDAB), lauryldimethylamine oxide (LDAO), an amine betaine (e.g., N,N-Dimethyl-N-dodecylglycine betaine (EMPIGEN BB)), or an ammonium sulfonate (e.g., n-Tetradecyl-N,N-dimethyl-3-ammonio-1-propanesulfonate (ZWITTERGENT 3-14)).

The crowding agent enhances loading of the reaction mixture into the partitions of a dPCR plate. The dPCR process assumes substantially even distribution of the reaction mixture and random partitioning of the target among the partitions of a dPCR plate. Disruptions to these assumptions therefore lower the reliability and accuracy of the dPCR results. For example, artifacts associated with the microfluidic loading of the multiple partitions can introduce unwanted and/or unknown variables that can negatively impact the results. The crowding agent is therefore intended to minimize such artifacts and promote even distribution and loading of the reaction mixture to the partitions of the dPCR plate.

In some embodiments, the crowding agent comprises one or more of polyethylene glycol (PEG), polyvinylpyrrolidone (PVP), dextran, bovine serum albumin (BSA), or an epichlorohydrin and sucrose copolymer (e.g., sold under the trade name Ficoll). In some embodiments, the crowding agent includes PEG, and the PEG has an average molecular weight of about 2,000 to about 14,000, or about 4,000 to about 12,000, or about 6,000 to about 10,000. In some embodiments, the crowding agent includes PVP, and the PVP has an average molecular weight of about 4,000 to about 14,000, or about 6,000 to about 12,000, or about 8,000 to about 11,000.

The crowding agent may be included at a concentration of about 0.5 mM to about 10 mM, or about 1 mM to about 7.5 mM, about 1 mM to about 5 mM, or within a range with endpoints defined by any two of the foregoing values. Including a crowding agent at such concentrations was found to beneficially enhance loading of the reaction mixture into the partitions of dPCR plates.

In some embodiments, the polymerase is a thermostable DNA polymerase, such as a Taq DNA polymerase, a mutant, variant, or derivative thereof. Various polymerases are known to the person skilled in the art and may be utilized accordingly. In some embodiments, the master mix also includes a reverse transcriptase to enable reverse transcription of target RNA to DNA or cDNA. The RNA target may include, for example, mRNA, miRNA or other types of small RNA, viral RNA targets, or other RNA targets.

In some embodiments, the master mix also includes one or more deoxynucleotide triphosphates (dNTPs). The master mix may include one or more of deoxythymidine triphosphate (dTTP), deoxyadenosine triphosphate (dATP), deoxycytidine triphosphate (dCTP), deoxyguanosine triphosphate (dGTP), 7-deaza-dGTP, or deoxyuridine triphosphate (dUTP). The master mix may additionally or alternatively include nucleotide analogs such as unnatural nucleotides, mutagen nucleotides, and/or fluorophore-labelled nucleotides. Each provided nucleotide may have a concentration of about 0.5 mM to about 5 mM, for example. The concentration of the individual dNTPs need not be identical. For example, dATP, dCTP, dGTP may be present in the composition at a first concentration while dUTP is present in the composition at a second, higher concentration.

In some embodiments, the master mix also includes a carrier. The carrier may include any formulation suitable for carrying the other components without significantly interfering with the intended amplification process. Examples include water, standard PCR buffer components, and polyols. In some embodiments, the carrier includes glycerol. The glycerol may be included at a concentration of about 5% to about 65% (v/v), or about 10% to about 60% (v/v), or about 20% to about 55% (v/v), or about 35% to about 50% (v/v), or within a range having endpoints defined by any two of the foregoing values.

In some embodiments, the master mix composition has a viscosity of about 1 cP to about 20 cP, or about 1 cP to about 15 cP, or about 1 cP to about 10 cP, or about 3 cP to about 8 cP, or a viscosity within a range with endpoints defined by any two of the foregoing values. Formulating the master mix with such a viscosity enables the beneficial microfluidic properties disclosed herein, such as even distribution to the dPCR plate.

In some embodiments, the master mix composition includes a reference control. The reference control may be an internal reference control that enables the user to establish a baseline fluorescent signal and/or determine distribution of the reaction mixture among the partitions of a dPCR plate. The reference control may be a dye such as a ROX dye, for example.

In some embodiments, the master mix composition includes a contamination control. For example, the master mix may include uracil DNA glycosylase (UNG). The UNG may be provided at a concentration such that its concentration in the final assembled PCR is about 0.005 to about 0.015 U/μL.

The master mix compositions described herein may also include other standard master mix components, such as buffer agents and/or salt solutions to provide appropriate pH and ionic conditions to maintain stability and effectiveness of the included polymerase enzymes. Examples of salt solutions include, for example, potassium chloride, potassium acetate, potassium sulfate, ammonium sulfate, ammonium chloride, ammonium acetate, magnesium chloride, magnesium acetate, magnesium sulfate manganese chloride, manganese acetate, manganese sulfate, sodium chloride, sodium acetate, lithium chloride and lithium acetate. It is to be understood that a wide variety of buffers and salt solutions are known in the art beyond those specifically disclosed herein.

In some embodiments, the master mix includes one or more components to enable “hot start” PCR. For example, the master mix may include an antibody, an aptamer, a hairpin primer, or a sequestration wax bead. Such components can be generated or selected using processes known in the art. Alternatively, a commercially available antibody can be used, for example, the TaqStart Antibody (Clontech) which is effective with any Taq-derived DNA polymerase, including native, recombinant, and N-terminal deletion mutants.

### Kits Including a Master Mix Composition

The master mix compositions described herein can be packaged in a suitable container capable of holding the composition and which will not significantly interact with components of the composition. The container can be one designed to permit easy dispensing of the dosage form by individuals or by a liquid handling instrument. The containers of composition can be further packaged into multi-pack units.

In some embodiments, the kit can include a control nucleic acid sample, a primer pair specific for amplification of at least a portion of the control nucleic acid sample, and/or a probe for detecting amplification of the control. Components of the kit other than the composition may be provided in individual containers or in a single container, as appropriate. Instructions and protocols for using the kit advantageously can be provided.

Kits can further comprise reagents used in one or more assays to synthesize, detect or quantify nucleic acids. In an embodiment, the kit can further comprise in addition to the composition a primer pair specific for amplification of a target nucleic acid and/or a probe specific for the nucleic acid target. For example, the probe can be a TaqMan® probe, a minor groove binding (MGB) probe, a locked nucleic acid (LNA) probe, or a cycling probe technology (CPT) probe. Additionally, or alternatively, one or more primers may include a detectable label.

Various probes are known in the art, for example TaqMan® probes (see, e.g., U.S. Pat. No. 5,538,848) various stem-loop molecular beacons (see, e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons (see, e.g., U.S. Pat. Nos. 6,355,421 and 6,593,091), linear PNA beacons (see, e.g., Kubista et al., 2001, SPIE 4264:53-58), non-FRET probes (see, e.g., U.S. Pat. No. 6,150,097), Sunrise®/Amplifluor® probes (U.S. Pat. No. 6,548,250), stem-loop and duplex Scorpion probes (see, e.g., Solinas et al., 2001, Nucleic Acids Research 29: E96 and U.S. Pat. No. 6,589,743), bulge loop probes (see, e.g., U.S. Pat. No. 6,590,091), pseudo knot probes (see, e.g., U.S. Pat. No. 6,589,250), cyclicons (see, e.g., U.S. Pat. No. 6,383,752), MGB Eclipse probe (Epoch Biosciences), hairpin probes (see, e.g., U.S. Pat. No. 6,596,490), peptide nucleic acid (PNA) light-up probes, self-assembled nanoparticle probes, and ferrocene-modified probes described, for example, in U.S. Pat. No. 6,485,901 and Mhlanga et al., 2001, Methods 25:463-471. Probes can also comprise two probes, wherein for example a fluor is on one probe, and a quencher on the other, wherein hybridization of the two probes together on a target quenches the signal, or wherein hybridization on a target alters the signal signature via a change in fluorescence.

Detector probes may be associated with quenchers such as dark fluorescent quencher (DFQ), black hole quenchers (BHQ), Iowa Black, QSY quencher, and Dabsyl and Dabcel sulfonate/carboxylate Quenchers. Detector probes may also include two probes, wherein, for example, a fluorophore is associated with one probe and a quencher is associated with a complementary probe such that hybridization of the two probes on a target quenches the fluorescent signal or hybridization on the target alters the signal signature via a change in fluorescence. Detector probes may also include sulfonate derivatives of fluorescein dyes with SO₃ instead of the carboxylate group, phosphoramidite forms of fluorescein, and/or phosphoramidite forms of Cy5, for example.

Other detectable labels may be used in addition to or as an alternative to labelled probes. For example, primers can be labeled and used to both generate amplicons and to detect the presence (or concentration) of amplicons generated in the reaction, and such may be used in addition to or as an alternative to labeled probes described herein. As a further example, primers may be labeled and utilized as described in Nazarenko et al. (Nucleic Acids Res. 2002 May 1; 30 (9): e37), Hayashi et al. (Nucleic Acids Res. 1989 May 11; 17 (9): 3605), and/or Neilan et al. (Nucleic Acids Res. Vol. 25, Issue 14, 1 Jul. 1997, pp. 2938-39). Those of skill in the art will also understand and be capable of utilizing the PCR processes (and associated probe and primer design techniques) described in Zhu et al. (Biotechniques. 2020 July: 10.2144/btn-2020-0057).

Exemplary detectable labels (e.g., for a probe and/or primer) include, for example, fluoresceins (e.g., 5-carboxy-2,7-dichlorofluorescein; 5-Carboxyfluorescein (5-FAM); 5-Hydroxy Tryptamine (5-HAT); 6-JOE; 6-carboxyfluorescein (6-FAM); Mustang Purple, VIC, ABY, JUN; FITC; 6-carboxy-4′,5′-dichloro-2′,7′-dimethoxy-fluorescein (JOE)); 6-carboxy-1,4-dichloro-2′,7′-dichloro-fluorescein (TET); 6-carboxy-1,4-dichloro-2′,4′,5′,7′-tetra-chlorofluorescein (HEX); Alexa Fluor fluorophores (e.g., 350, 405, 430, 488, 500, 514, 532, 546, 555, 568, 594, 610, 633, 635, 647, 660, 680, 700, 750); BODIPY fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), Cascade Blue, Cascade Yellow; Cy dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalama1), cyan fluorescent protein (e.g., ECFP, Cerulean, CyPet), yellow fluorescent protein (e.g., YFP, Citrine, Venus, YPet)), FRET donor/acceptor pairs (e.g., fluorescein/fluorescein, fluorescein/tetramethylrhodamine, IAEDANS/fluorescein, EDANS/dabcyl, BODIPY FL/BODIPY FL, Fluorescein/QSY7 and QSY9), LysoTracker and LysoSensor (e.g., LysoTracker Blue DND-22, LysoTracker Blue-White DPX, LysoTracker Yellow HCK-123, LysoTracker Green DND-26, LysoTracker Red DND-99, LysoSensor Blue DND-167, LysoSensor Green DND-189, LysoSensor Green DND-153, LysoSensor Yellow/Blue DND-160, LysoSensor Yellow/Blue 10,000 MW dextran), Oregon Green (e.g., 488, 488-X, 500, 514); rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethyl-rhodamine), Tetramethylrhodamine (TRITC), WT), Texas Red, Texas Red-X, among others as known to those of skill in the art.

In some embodiments, intercalating labels can be used such as ethidium bromide, SYBR Green I, SYBR GreenER, and PicoGreen (Life Technologies Corp., Carlsbad, CA), thereby allowing visualization in real-time, or end point, of an amplification product.

The disclosed compositions and kits can be used in a variety of amplification assays to detect or quantify nucleic acid. For example, the compositions can be used in gene expression assays (e.g., TaqMan® Gene Expression Assays), miRNA assays (e.g., TaqMan® MicroRNA Assays), genotyping assays (e.g., TaqMan® Drug Metabolism Genotyping Assays or TaqMan® SNP Genotyping Assays), pathogen detection assays, and/or RNA quantitation assays (e.g., two-step reverse transcription-polymerase chain reaction assays).

### Methods of Nucleic Acid Amplification

In one embodiment, a method for amplifying a nucleic acid molecule comprises: (a) mixing a composition with a nucleic acid sample (that may include one or more target nucleic acids), the composition including (i) at least one DNA polymerase, (ii) at least one crowding agent, and (iii) at least one surfactant at a concentration of at least 0.05% (w/v); and (b) incubating the mixture under conditions sufficient to amplify a nucleic acid molecule complementary to all or a portion of said nucleic acid template. The composition may be any of the master mix composition embodiments described herein.

Examples of reaction vessels in which amplification is performed include microcentrifuge tubes, wells of a wellplate, capillary tubes, microfluidic chips, and/or partitions within a dPCR plate.

In some embodiments, the amplification method includes dPCR. In some embodiments, the composition includes at least one reverse transcriptase, and the amplification method includes RT-PCR. In some embodiments, the method includes one-step RT-PCR (e.g., in a single vessel or reaction volume) in which one or more reverse transcriptases are used in combination with one or more DNA polymerases. Some embodiments implement both RT-PCR (e.g., one-step) and dPCR. For example, some embodiments utilize one or more reverse transcriptases in combination with one or more DNA polymerases in a dPCR instrument and system to provide dPCR output.

In some dPCR embodiments, the method includes filling at least 80%, 85%, 90%, 95%, 96%, 97%, 98%, or 99% of the reaction chambers (i.e., the partitions) with the reaction mixture. The partitions may have a total volume of between 5 and 15 microliters, between 5 and 12 microliters, between 5 and 10 microliters, between 5 and 9 microliters, between 5 and 8 microliters, between 5 and 7 microliters, or between 5 and 6 microliters, for example.

In some dPCR embodiments, the method beneficially includes partitioning the reaction mixture such that at least 80%, 85%, 90%, 95%, 96%, 97%, 98%, or 99% of the reaction chambers retain DNA polymerase activity and/or such that at least 80%, 85%, 90%, 95%, 96%, 97%, 98%, or 99% of the reaction chambers are positive for the reference control upon testing for the reference control (e.g., ROX). Typical dPCR reactions include more than 20,000 partitions.

In some dPCR embodiments, the nucleic acid sample comprises genomic DNA with a target locus having a mutant allele frequency of 1% or less, 0.75% or less, 0.5% or less, 0.25% or less, 0.15% or less, or 0.1% or less, and the method is capable of detecting the mutant allele when present within the nucleic acid sample. In some embodiments, the method is capable of detecting the mutant allele in the presence of an amplification inhibitor such as hematin. In some embodiments, the hematin is present at a concentration of about 10 μM to about 100 μM, such as about 50 μM.

Sources for nucleic acid samples include, but are not limited to, for example clothing, soil, paper, metal surfaces, air, water, plant parts, as well as human and/or animal skin, hair, blood, serum, feces, milk, saliva, urine, and/or other secretory fluids. These sources may also contain compounds (e.g., hematin) that inhibit PCR amplification.

Amplification methods may be singleplex or multiplex. As a non-limiting example, a 4-plex reaction can include FAM (emission peak ˜517 nm), VIC (emission peak ˜551 nm), ABY (emission peak ˜580 nm), and JUN (emission peak ˜617 nm) dyes. In some embodiments, each dye is associated with one or more target sequences. In some embodiments, up to 2, 4, 6, 8, 10, or 12 targets are amplified and tracked real-time within a single reaction vessel, using any combination of detectable labels disclosed herein or otherwise known to those of skill in the art. The aforementioned reporter dyes are optimized to work together with minimal spectral overlap for improved performance. Any combination of dyes described herein can additionally be combined with other dyes (e.g., Mustang Purple (emission peak ˜654 nm) or one or more Alexa Fluors (e.g., AF647 and AF676)), for use in monitoring fluorescence of a control or for detecting additional or alternative targets. In some multiplex embodiments, at least one of the targets is an endogenous or exogenous internal positive control (e.g., RNase P).

In general, PCR thermal cycling includes an initial denaturing step at high temperature, followed by a repetitive series of temperature cycles designed to allow template denaturation, primer annealing, and extension of the annealed primers by the polymerase. Generally, the samples are heated initially for about 2 to 10 minutes at a temperature of about 95° C. to denature the double stranded DNA sample. Then, in the beginning of each cycle, the samples are denatured for about 10 to 60 seconds, depending on the samples and the type of instrument used. After denaturing, the primers are allowed to anneal to the target DNA at a lower temperature, from about 40° C. to about 60° C. for about 10 to 60 seconds. Extension of the primers by the polymerase is often carried out at a temperature ranging from about 60° C. to about 72° C. The amount of time used for extension will depend on the size of the amplicon and the type of enzymes used for amplification and is readily determined by routine experimentation. Additionally, the annealing step can be combined with the extension step, resulting in a two-step cycling. Thermal cycling may also include additional temperature shifts in PCR assays. The number of cycles used in the assay depends on many factors, including the primers used, the amount of sample DNA present, and the thermal cycling conditions. The number of cycles to be used in any assay may be readily determined by one skilled in the art using routine experimentation. Optionally, a final extension step may be added after the completion of thermal cycling to ensure synthesis of all amplification products.

### Selected List of Terms & Definitions

As used herein, “nucleic acid” includes compounds having a plurality of natural nucleotides and/or non-natural (or “derivative”) nucleotide units. A “nucleic acid” can further comprise non-nucleotide units, for example peptides. “Nucleic acid” therefore encompasses compounds such as DNA, RNA, peptide nucleic acids, phosphothioate-containing nucleic acids, phosphonate-containing nucleic acids and the like. There is no particular limit as to the number of units in a nucleic acid, provided that the nucleic acid contains 2 more nucleotides, nucleotide derivatives, or combinations thereof, specifically 5, 10, 15, 25, 50, 100, or more. Nucleic acids can encompass both single and double-stranded forms, and fully or partially duplex hybrids (e.g., RNA-DNA, RNA-PNA, or DNA-PNA).

The term “primer” may refer to more than one primer and refers to an oligonucleotide, whether occurring naturally, as in a purified restriction digest, or produced synthetically, which is capable of acting as a point of initiation of synthesis along a complementary strand when placed under conditions in which synthesis of a primer extension product which is complementary to a nucleic acid strand is catalyzed. Such conditions include the presence of four different deoxyribonucleoside triphosphates and a polymerization-inducing agent such as DNA polymerase or reverse transcriptase, in a suitable buffer and at a suitable temperature. A primer is typically 11 bases or longer; more specifically, a primer is 17 bases or longer, although shorter or longer primers may be used depending on particular application needs.

As used herein, the term “target”, “target sequence” or “target nucleic acid sequence” refers to a region of a nucleic acid which is to be either amplified, detected, or both. The target sequence resides between the two primer sequences used for amplification.

The term “label” as used herein refers to any atom or molecule which can be used to provide a detectable signal, and which can be attached to a nucleic acid or protein. Labels may provide signals detectable by fluorescence, radioactivity, colorimetry, gravimetry, X-ray diffraction or absorption, magnetism, enzymatic activity, and the like. In some embodiments, the detectable signal is a quantifiable signal.

Unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

### EXAMPLES

### Example 1: Protocol for Digital POR

A dPCR protocol was developed using a QuantStudio® Absolute Q® Digital PCR System and QuantStudio® Absolute Q® MAP16 Digital PCR Plate Kit. Materials utilized are listed in Table 1.

TABLE 1
Materials

| | |
|---|---|
| Item | Supplier |

Equipment

| | |
|---|---|
| Vortex Mixer | MLS* |
| Single and Multichannel pipettes | MLS |
| capable of pipetting up to 2, 10, 20, | |
| 50, 100, 200, and 1000 μl volumes | |
| Mini centrifuge | MLS |
| Absolute Q ® Digital PCR System | Thermo Fisher Scientific |
| Centrifuge | MLS |

Reagents and Consumables

| | |
|---|---|
| 70% Isopropanol spray or wipes | MLS |
| 1.5 mL Microcentrifuge Tube | MLS |
| (DNAse and RNAse Free) | |
| MAP16 Digital PCR Plate | Applied Biosystems |
| MAP16 Gaskets Strips, 50 pack (5 | Applied Biosystems |
| per plate) | |
| QuantStudio ® Absolute Q ® Digital | Thermo Fisher Scientific |
| PCR Starter Kit | |
| Absolute Q ® Digital PCR Starter | Thermo Fisher Scientific |
| Assay, 20X | |
| Absolute Q ® DNA dPCR Mix (5X) | Thermo Fisher Scientific |
| CEPH DNA | Thermo Fisher Scientific |
| QuantStudio Absolute Q Isolation | Applied Biosystems |
| Buffer | |
| Nuclease free water | Thermo Fisher Scientific |

Software

| | |
|---|---|
| QuantStudio Absolute Q Digital | Thermo Fisher Scientific |
| PCR Software | |

*MLS = generally available from major laboratory suppliers

The Absolute Q® DNA dPCR Mix (5×) is one example of a master mix composition as described herein. The Absolute Q® DNA dPCR Mix (5×) included the components shown in Table 2 in amounts within the ranges described in this disclosure. The composition may occasionally be referred to in these Examples as simply “the master mix.”

TABLE 2
Absolute Q ® DNA dPCR Mix (5X) Selected Components
Component

Carrier
Crowding agent
Surfactant/Detergent
ROX passive reference standard
AmpliTaq ® Polymerase

Testing Setup: The Absolute Q® DNA dPCR Mix (5×), Starter Kit (20×) and CEPH DNA sample were thawed, then spun down via benchtop centrifuge prior to use. The same lot of nuclease free water was prepared and used throughout the course of sample preparation.

Preparation of Intermediate Dilution: A 1.5 mL tube was labelled as intermediate dilution. Subsequently, 36 μL of nuclease free water was transferred into the 1.5 mL tube via single channel pipette. Then, 4 μL of the CEPH DNA (stock concentration of 50 ng/μL, as labeled) was added to the 1.5 mL tube. The tube was vortexed and then spun down via benchtop centrifuge. The resulting intermediate dilution had a concentration of 5 ng/μL.

Preparation of Reaction Mixture: Three 1.5 mL tubes were respectively labelled NAC (no assay control), NTC (no template control), and POS (includes assay and template). The reaction mixes for these tubes were prepared according to Table 3. Reaction volumes for 1 and 16 reactions are detailed in Table 4. The NAC, NTC, and POS tubes were vortexed and spun down via benchtop centrifuge.

TABLE 3
Reaction Mix Preparation

| | Final | NAC | NTC | POS |
|---|---|---|---|---|
| Reagent | Concentration | (μL) | (μL) | (μL) |
| 5x Master Mix | 1x | 8 | 8 | 16 |
| Absolute Q ® Digital PCR | 1x | — | 2 | 4 |
| Starter Assay, 20X | | | | |
| 5 ng/μL CEPH DNA | 10 ng/9 μL | 8.9 | — | 17.8 |
| Intermediate Dilution | | | | |
| Nuclease Free Water | Fill to 10 μL | 23.1 | 30 | 42.2 |

| | | | |
|---|---|---|---|
| Total + 10% Overage (in μL) | 40 | 40 | 80 |

TABLE 4
Total Reaction Volumes

| | | Volume for | Volume for |
|---|---|---|---|
| | Final | 1 rxn | 16 rxns |
| Reagent | Concentration | (μL) | (μL) |
| 5x Master Mix | 1x | 2 | 32 |
| Absolute Q ® Digital | 1x | 0.5 | 8 |
| PCR Starter Assay, 20X | | | |
| 5 ng/μL CEPH DNA | 10 ng/9 μL | 2.2 | 35.5 |
| Intermediate Dilution | | | |
| Nuclease Free Water | — | 5.3 | 84.5 |

| | | |
|---|---|---|
| Total + 10% Overage (in μL) | 10 | 160 |

Sample Loading to Plate: The reaction mixtures were transferred to corresponding wells on the MAP16 plate at 9 μL per well (4 wells NAC, 4 wells NTC, 8 wells POS). 15 μL of isolation buffer was then transferred to each loaded well. Gaskets were then placed on the plate in accordance with manufacturer instructions.

Loading Plate to Instrument: The plate was loaded into the QuantStudio® Absolute Q® Digital PCR System. The thermal cycling protocol was setup with a 10 minute preheat period to 96° C. followed by 40 cycles of 96° C. for 5 seconds and 60° C. for 15 seconds. Targets were successfully analyzed using the dye/target combinations shown in Table 5.

TABLE 5
Dyes and Corresponding Targets

| Dye | Target |
|---|---|
| FAM | Target 1 |
| VIC | Target 2 |
| ABY | Control |
| JUN | Target 1 and 2 |

Except as noted in the specific details below, the following Examples utilized similar materials and followed a similar process to that outlined above for Example 1.

### Example 2: Master Mix Component Optimization

The detergent, polymerase, and dNTP concentrations were varied from nominal starting concentrations with the goal of decreasing the failure rate. Passing criteria required a mean positive signal that is greater than 90% of nominal; a resolution greater than two, and total valid partitions per array of greater than 20,000 (out of 20,480 total partitions). The DNA sample utilized cDNA made from Universal Human Reference RNA (Agilent, Part No. 74000) using the High-Capacity Reverse Transcription Kit (Thermo Fisher Scientific, Part No. 4387406). The cDNA was included at 0.2 ng/μL in the reaction. Custom formulated Xeno DNA was also included at 5 cp/μL. A suitable alternative for the Xeno DNA described in these Examples is VetMAX™ Xeno™ Internal Positive Control DNA, available from Thermo Fisher Scientific, Catalog No. A29762. The assay utilized was a 4-plex gene expression assay with the dye/target combinations shown in Table 6.

TABLE 6
Dyes and Corresponding Targets

| Dye | Target |
|---|---|
| FAM | CYC1 |
| VIC | Xeno |
| ABY | CD44 |
| CY5 | G6PD |

Results showed that adjusting the detergent concentration to 0.11% (w/v) and polymerase concentration to 1.1 U/μL would beneficially reduce the failure rate to 1.4%.

### Example 3: Detection of a Low Abundance Target in a Multiplex Reaction

To determine that the master mix could detect a low abundance target in a multiplex reaction, Xeno DNA was added at 1 copy/μL while the other targets (CD44, CYC1, G6PD) were added at inputs of 500 to 3,000 copies/μL. Table 7 shows that detection of the Xeno DNA was successfully achieved at all input concentrations of the other targets.

TABLE 7
Xeno DNA detection at Different Levels of Other Targets

| Input of Other | | | | |
|---|---|---|---|---|
| Targets* | Mean | Std. Dev. | Min. | Max. |
| 500 | 0.82 | 0.25 | 0.47 | 1.05 |
| 1,000 | 0.94 | 0.37 | 0.59 | 1.41 |
| 2,000 | 0.85 | 0.06 | 0.82 | 0.94 |
| 3,000 | 0.80 | 0.45 | 0.35 | 1.41 |

*Xeno DNA input = 1 copy/μL

### Example 4: Detection of Low Abundance Target with High Confidence

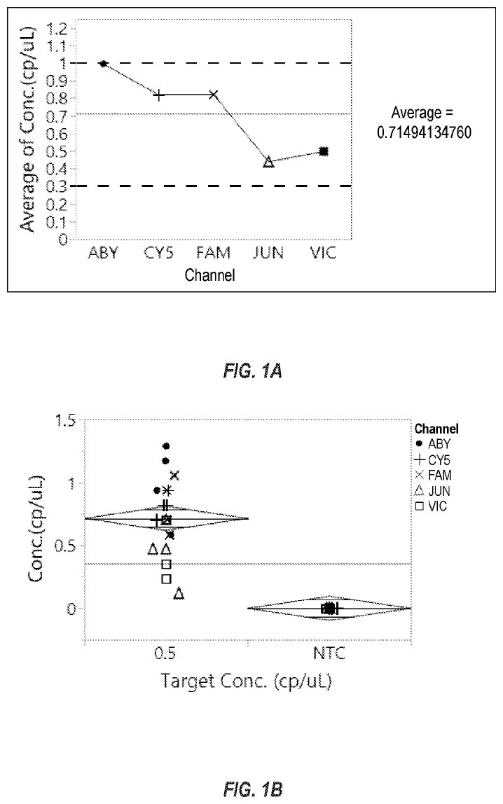

The master mix was tested to determine whether a low abundance target (0.5 copies/μL) could be detected with 95% confidence relative to the NTC (no template control). FIG. 1A illustrates the average measured concentrations of 0.5 copy/μL Xeno DNA for ABY, CY5, FAM, JUN, and VIC dyes (from corresponding probes labelled with these dyes). FIG. 1B illustrates one-way analysis of variance of measured concentration by target concentration (0.5 copies/μL or NTC). The means diamonds represent 95% confidence intervals for the respective means. The p-values for each dye are shown in Table 8. Results illustrate that measured values of the low abundance target, when input at 0.5 copies/μL, were different to a statistically significant degree from the NTC.

TABLE 8
p-values Compared to NTC for Various Dyes

| | Average Measured Result | p-value Compared |
|---|---|---|
| Dye | for 0.5 copies/μL input | to NTC |
| ABY | 1.00 | 0.0077 |
| CY5 | 0.82 | 0.0004 |
| FAM | 0.82 | 0.0046 |
| JUN | 0.44 | 0.0358 |
| VIC | 0.50 | 0.0259 |

### Example 5: Detection of Low Frequency Mutant Allele

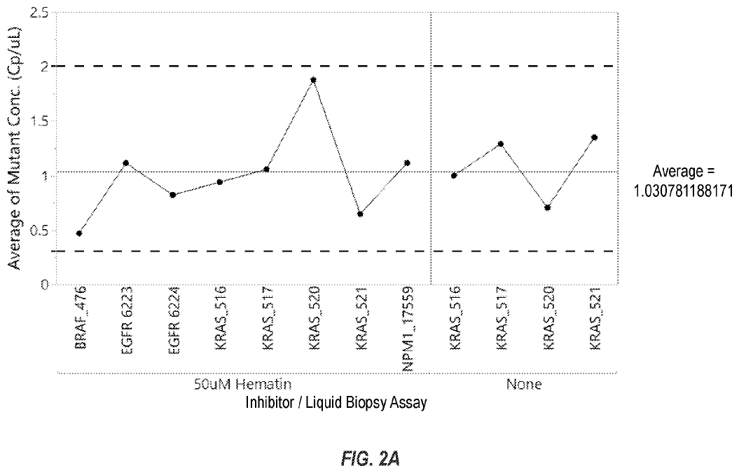

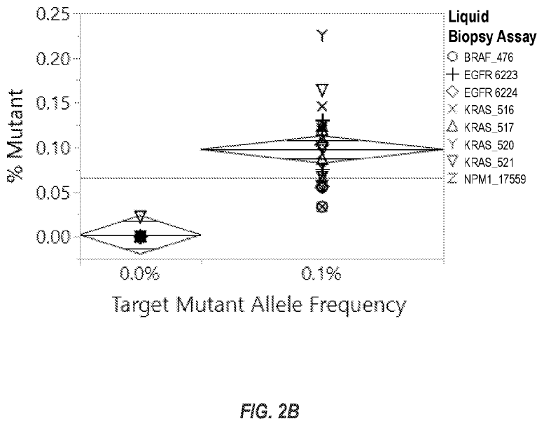

The master mix was tested to determine whether a rare mutant allele could be detected in the presence of an inhibitor. Target mutant alleles were provided at 0.1% in a background of 30 ng/reaction human genomic DNA (Control DNA (from CEPH Individual 1347-02), available from Thermo Fisher Scientific, Catalog No. 403062). Some target samples further included 50 μM/reaction hematin. The goal was for ≥80% of Qualified rare mutation oncology assays to detect 0.5-1.5 copies/μL of mutant allele in the 30 ng/reaction human genomic DNA background. FIG. 2A illustrates the average measured concentrations (based on FAM signal) for various rare allele targets, some in the presence of 50 μM/reaction hematin and some without. FIG. 2B illustrates one-way analysis of variance of measured % mutant alleles by target concentration (0.1% mutant alleles or 0% mutant alleles). The means diamonds represent 95% confidence intervals for the respective means. A t-test of the results provided a p-value of <0.0001.

### Example 6: Identification of Amplified and Unamplified Partitions

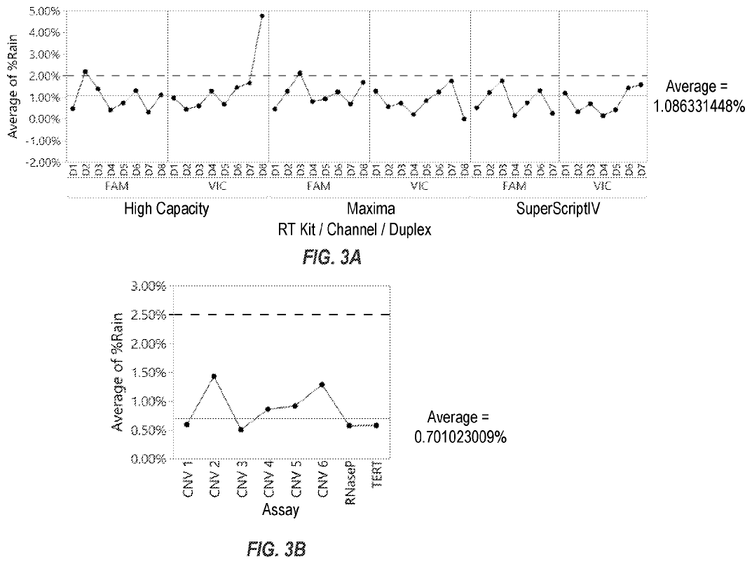

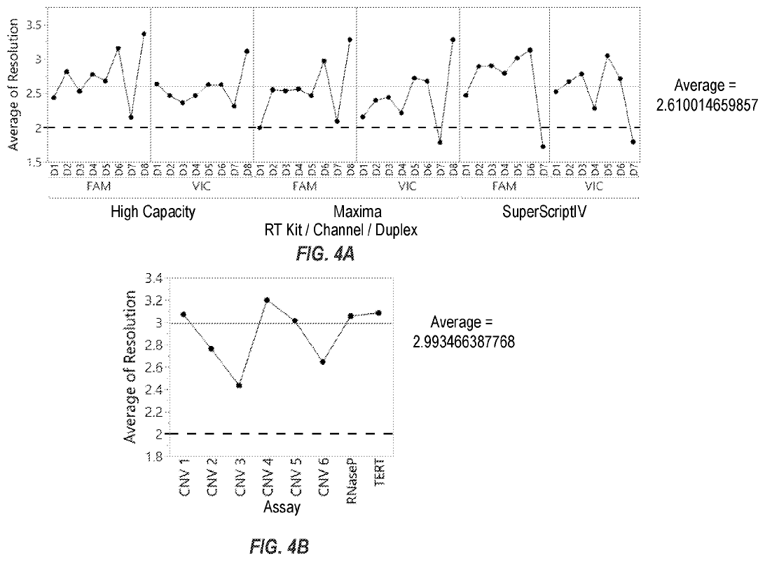

The master mix was tested to determine percent rain and resolution for assays with DNA input from 100-4,000 copies/μL. The input DNA was cDNA generated from Universal Reference RNAs (Agilent, Part No. 740000). FIG. 3A shows average of percent rain (i.e., intermediate fluorescence) for various assay kits, corresponding dye channels (FAM and VIC), and duplex replicates. The assay kits were High-Capacity Reverse Transcription Kit (Thermo Fisher Scientific, Part No. 4387406) (“High Cap”), Maxima (Part No. M1682), and Superscript IV Vilo (Part No. 1766500). FIG. 3B shows average percent rain for various particular targets. As shown, essentially all tests resulted in a percent rain well below the 2.5% threshold goal. FIGS. 4A and 4B show average resolution for the same assays, showing that essentially all tests provided a resolution above the threshold goal of two.

### Example 7: Partition Passive Reference Testing

The proportion of total valid partitions of the dPCR Plate, the mean ROX signal per well, and the ROX signal per well % CV were measured. Criteria for passing were to achieve greater than 20,000 analyzed partitions per well, a mean ROX signal per well >6,000 at least 95% of the time, and a ROX signal per well coefficient of variation (% CV) less than 10.5% at least 95% of the time when assay is run without JUN dye. Results are summarized in Table 9. The results show that each of these criteria were achieved.

TABLE 9
Partition Testing

| Metric | N | Pass Rate |
|---|---|---|
| Total Valid Partitions | 262 | 97.7% |
| Mean ROX | 257 | 99.2% |
| ROX % CV | 209 | 96.7% |

### Example 8: Detergent Concentration Testing

A series of dPCR runs were performed to test different surfactant concentrations in the master mix. Results are illustrated in FIG. 5, where triangles represent partitions above the fluorescence threshold (positive/true) and circles represent partitions below the threshold (negative/false). As shown, when the master mix includes lower concentrations of surfactant, there was insufficient detection of positive partitions and/or inability to effectively distinguish between positive and negative partitions because many measured signals were close to the threshold. Levels at and above about 0.015% (w/v) enabled effective identification of positive partitions and ability to distinguish positive and negative partitions. As discussed above in Example 2, even higher levels of surfactant (e.g., 0.11% (w/v)) were found to further promote effective dPCR results.

FIG. 6 compares images showing positive partitions (bright spots) of a dPCR plate where the master mix included sufficient surfactant and a dPCR plate where the master mix included insufficient surfactant. As shown, the plate with insufficient surfactant includes “dead” regions without positive partitions, whereas the plate with sufficient surfactant provides well distributed positive partitions, as would be expected for a random distribution of target nucleic acids.

### Example 9: Crowding Agent Testing

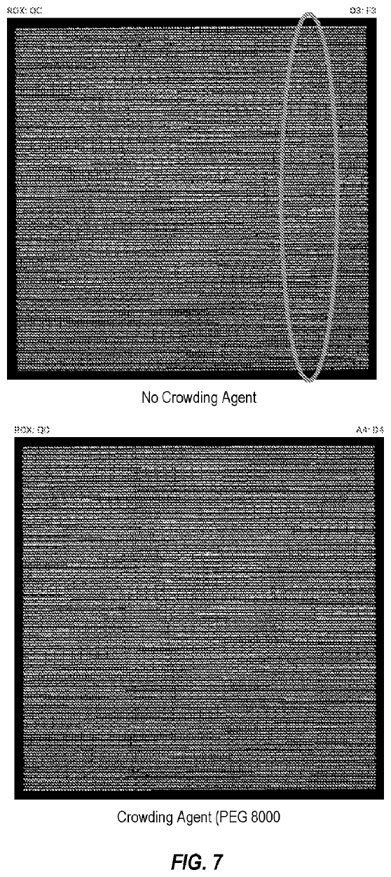

FIG. 7 compares images showing the passive reference signal (ROX) for a dPCR plate with master mix that did not include a crowding agent to a dPCR plate with master mix that did include a crowding agent (0.5% PEG 8000). As shown, the use of a crowding agent evened plate loading and eliminated or minimized reagent loading artifacts such as the highlighted band shown in the no-crowding agent image.

1. A composition comprising:
at least one DNA polymerase;
at least one crowding agent; and
at least one least one surfactant, wherein the concentration of the surfactant is at least 0.05% (w/v) or greater.

2. The composition of claim 1, wherein the concentration of the surfactant is at least 0.055%, 0.06%, 0.065%, 0.07%, 0.075%, 0.08%, 0.085%, 0.09%, 0.095%, 0.1%, 0.105%, or 0.11% (w/v) or greater.

3. The composition of claim 1 or claim 2, wherein the concentration of the surfactant is between 0.05% and 0.2%, or between 0.06% and 0.18%, or between 0.07% and 0.16%, or between 0.07% and 0.15%, or between 0.08% and 0.125%, or between 0.09% and 0.12%.

4. The composition of any one of claims 1-3, wherein the crowding agent comprises one or more of polyethylene glycol (PEG), polyvinylpyrrolidone (PVP), dextran, bovine serum albumin (BSA), or an epichlorohydrin and sucrose copolymer (Ficoll).

5. The composition of claim 4, wherein the crowding agent comprises PEG and the PEG has an average molecular weight of about 2,000 to about 14,000, or about 4,000 to about 12,000, or about 6,000 to about 10,000.

6. The composition of claim 4 or claim 5, wherein the crowding agent comprises PVP with an average molecular weight of about 4,000 to about 14,000, or about 6,000 to about 12,000, or about 8,000 to about 11,000.

7. The composition of any one of claims 1-6, wherein the concentration of the crowding agent is between 1 mM and 10 mM.

8. The composition of claim 7, wherein the concentration of the crowding agent is between 1 mM and 5 mM.

9. The composition of any one of claims 1-8, wherein the surfactant comprises a nonionic surfactant.

10. The composition of claim 9, wherein the surfactant comprises one or more of polysorbate 20 (TWEEN 20), polysorbate 80 (TWEEN 80), 2-[4-(2,4,4-trimethylpentan-2-yl)phenoxy]ethanol (Triton X-100), an alcohol ethoxylate (e.g., Ecosurf EH-9, TERGITOL 15-S-9), nonyl phenoxypolyethoxylethanol (NP-40), 3-((3-cholamidopropyl) dimethylammonio)-1-propanesulfonate (CHAPS), 3-[(3-cholamidopropyl)dimethylammonio]-2-hydroxy-1-propanesulfonate (CHAPSO), polyoxyethylene (23) lauryl ether (Brij-35), polyoxyethylene (20) cetyl ether (Brij-58), polyoxyethylene (20) stearyl ether (Brij-78), or polyoxyethylene (20) oleyl ether (Brij-98).

11. The composition of any one of claims 1-10, wherein the surfactant comprises a cationic or zwitterionic surfactant.

12. The composition of claim 11, wherein the surfactant comprises one or more of benzalkonium chloride (BZK), didodecyldimethylammonium bromide (DDAB), lauryldimethylamine oxide (LDAO), an amine betaine (e.g., N,N-Dimethyl-N-dodecylglycine betaine (EMPIGEN BB)), or an ammonium sulfonate (e.g., n-Tetradecyl-N,N-dimethyl-3-ammonio-1-propanesulfonate (ZWITTERGENT 3-14)).

13. The composition of any one of claims 1-12, wherein the composition is configured for use in digital PCR (dPCR), reverse transcription PCR (RT-PCR), or quantitative/real-time PCR (qPCR).

14. The composition of any one of claims 1-13, wherein said DNA polymerase is a thermostable DNA polymerase.

15. The composition of claim 14, wherein said thermostable DNA polymerase is Taq DNA polymerase, a mutant, variant, or derivative thereof.

16. The composition of any one of claims 1-15, further comprising one or more nucleotides (dNTPs).

17. The composition of claim 16, wherein said nucleotides are selected from the group consisting of dTTP, dATP, dCTP, dGTP, 7-deaza-dGTP, or dUTP.

18. The composition of claim 16 or 17, wherein the concentration of each of said nucleotides is about 0.5 mM to 5 mM.

19. The composition of any one of claims 1-18, further comprising a carrier.

20. The composition of claim 19, wherein the carrier is glycerol.

21. The composition of claim 20, wherein the concentration of said glycerol is between 5%-50% (v/v).

22. The composition of any one of claims 1-21, further comprising a reference control.

23. The composition of claim 22, wherein the reference control is a dye.

24. The composition of claim 23, wherein the reference control is a ROX dye.

25. The composition of any one of claims 1-24, wherein the composition is a concentrated stock solution.

26. The composition of claim 25, wherein said concentrated stock solution is a 2× to 5× stock solution.

27. The composition of any one of claims 1-26, wherein the viscosity of the composition ranges between about 1 cP and about 20 cP.

28. The composition of claim 27, wherein the viscosity of the composition ranges between about 1 cP and about 15 cP.

29. The composition of claim 28, wherein the viscosity of the composition ranges between about 1 cP and about 10 cP.

30. The composition of claim 29, wherein the viscosity of the composition ranges between about 3 cP and about 8 cP.

31. A method of performing digital polymerase chain reaction (dPCR) of a nucleic acid sample, the method comprising:
(a) mixing a composition with a nucleic acid sample to form a reaction mixture, the composition comprising:
at least one DNA polymerase,
at least one crowding agent,
at least one surfactant at a concentration of at least 0.05% (w/v), and
at least one reference control;
(b) partitioning the mixture to a plurality of reaction chambers such that the reference control is partitioned into at least 80%, 85%, 90%, 95%, 96%, 97%, 98%, or 99% of the reaction chambers; and
(c) subjecting the nucleic acid sample in each of the reaction chambers to amplification conditions.

32. The method of claim 31, wherein said nucleic acid sample is selected from the group consisting of blood, sweat, tears, soil, saliva, urine, and feces.

33. The method of any one of claims 31-32, wherein the method further comprises filling at least 80%, 85%, 90%, 95%, 96%, 97%, 98%, or 99% of the reaction chambers with the reaction mixture, wherein the reaction chambers have a total volume of between 5 and 15 microliters, between 5 and 12 microliters, between 5 and 10 microliters, between 5 and 9 microliters, between 5 and 8 microliters, between 5 and 7 microliters, or between 5 and 6 microliters.

34. The method of any one of claims 31-33, wherein the mixture is partitioned such that at least 80%, 85%, 90%, 95%, 96%, 97%, 98%, or 99% of the reaction chambers retain DNA polymerase activity.

35. The method of any one of claims 31-34, wherein the mixture is partitioned such that at least 80%, 85%, 90%, 95%, 96%, 97%, 98%, or 99% of the reaction chambers are positive for the reference control upon testing for the reference control.

36. The method of any one of claims 31-35, wherein the nucleic acid sample comprises genomic DNA with a target locus having a mutant allele frequency of 1% or less, 0.75% or less, 0.5% or less, 0.25% or less, 0.15% or less, or 0.1% or less, and wherein the method is capable of detecting the mutant allele when present within the nucleic acid sample.

37. The method of claim 36, wherein the method is capable of detecting the mutant allele in the presence of an amplification inhibitor.

38. The method of claim 37, wherein the amplification inhibitor is hematin.

39. The method of claim 38, wherein the hematin is present at a concentration of up to about 50 μM.

40. The method of any one of claims 31-39, wherein the nucleic acid sample comprises a nucleic acid target at a concentration of 1 cp/μL or less, 0.9 cp/μL or less, 0.8 cp/μL or less, 0.7 cp/μL or less, 0.6 cp/μL or less, or 0.5 cp/μL or less, and wherein the method is capable of detecting the nucleic acid target with 95% confidence or greater.

41. The method of any one of claims 31-39, wherein the concentration of the surfactant is at least 0.055%, 0.06%, 0.065%, 0.07%, 0.075%, 0.08%, 0.085%, 0.09%, 0.095%, 0.1%, 0.105%, or 0.11% (w/v) or greater.

42. The method of any one of claims 31-41, wherein the concentration of the surfactant is between 0.05% and 0.2%, or between 0.06% and 0.18%, or between 0.07% and 0.16%, or between 0.07% and 0.15%, or between 0.08% and 0.125%, or between 0.09% and 0.12%.

43. The method of any one of claims 31-42, wherein the crowding agent comprises one or more of polyethylene glycol (PEG), polyvinylpyrrolidone (PVP), dextran, bovine serum albumin (BSA), or an epichlorohydrin and sucrose copolymer (Ficoll).

44. The method of claim 43, wherein the crowding agent comprises PEG and the PEG has an average molecular weight of about 2,000 to about 14,000, or about 4,000 to about 12,000, or about 6,000 to about 10,000.

45. The method of claim 43 or 44, wherein the crowding agent comprises PVP with a molecular weight of about 4,000 to about 14,000, or about 6,000 to about 12,000, or about 8,000 to about 11,000.

46. The method of any one of claims 31-45, wherein the concentration of the crowding agent is between 1 mM and 10 mM.

47. The method of claim 46, wherein the concentration of the crowding agent is between 1 mM and 5 mM.

48. The method of any one of claims 31-47, wherein the surfactant is a nonionic surfactant.

49. The method of claim 48, wherein the surfactant comprises one or more of polysorbate 20 (TWEEN 20), polysorbate 80 (TWEEN 80), 2-[4-(2,4,4-trimethylpentan-2-yl)phenoxy]ethanol (Triton X-100), an alcohol ethoxylate (e.g., Ecosurf EH-9, TERGITOL 15-S-9), nonyl phenoxypolyethoxylethanol (NP-40), 3-((3-cholamidopropyl) dimethylammonio)-1-propanesulfonate (CHAPS), 3-[(3-cholamidopropyl)dimethylammonio]-2-hydroxy-1-propanesulfonate (CHAPSO), polyoxyethylene (23) lauryl ether (Brij-35), polyoxyethylene (20) cetyl ether (Brij-58), polyoxyethylene (20) stearyl ether (Brij-78), or polyoxyethylene (20) oleyl ether (Brij-98).

50. The method of any one of claims 31-49, wherein the surfactant comprises a cationic or zwitterionic surfactant.

51. The method of claim 50, wherein the surfactant comprises one or more of benzalkonium chloride (BZK), didodecyldimethylammonium bromide (DDAB), lauryldimethylamine oxide (LDAO), an amine betaine (e.g., N,N-Dimethyl-N-dodecylglycine betaine (EMPIGEN BB)), or an ammonium sulfonate (e.g., n-Tetradecyl-N,N-dimethyl-3-ammonio-1-propanesulfonate (ZWITTERGENT 3-14)).

52. The method of any one of claims 31-51, wherein said DNA polymerase is a thermostable DNA polymerase.

53. The method of claim 52, wherein said thermostable DNA polymerase Taq DNA polymerase or a mutant, variant, or derivative thereof.

54. The method of any one of claims 31-53, further comprising one or more nucleotides (dNTPs).

55. The method of claim 54, wherein said nucleotides are selected from the group consisting of dTTP, dATP, dCTP, dGTP, 7-deaza-dGTP, or dUTP.

56. The method of claim 54 or 55, wherein the concentration of each of said nucleotides is about 0.5 mM to 5 mM.

57. The method of any one of claims 31-56, further comprising a carrier.

58. The method of claim 57, wherein the carrier is glycerol.

59. The method of claim 58, wherein the concentration of said glycerol is between 5%-50%.

60. The method of any one of claims 31-59, further comprising a reference control.

61. The method of claim 60, wherein the reference control is a dye.

62. The method of claim 61, wherein the reference control is a ROX dye.

63. The method of any one of claims 31-62, wherein said composition is a concentrated stock solution.

64. The method of claim 63, where said concentrated stock solution is a 2× to 5× stock solution.

65. The method of any one of claims 31-64, wherein the viscosity of the composition ranges between about 1 cP to about 20 cP, or about 1 cP to about 15 cP, or about 1 cP to about 10 cP, or about 3 cP to 8 cP.

66. A method for amplifying a nucleic acid molecule, said method comprising:
(a) mixing a composition with a nucleic acid sample, the composition comprising:
at least one DNA polymerase,
at least one crowding agent, and
at least one surfactant at a concentration of at least 0.05% (w/v); and
(b) incubating the mixture under conditions sufficient to amplify a nucleic acid molecule complementary to all or a portion of said nucleic acid template.

67. The method of claim 66, wherein the concentration of the surfactant is at least 0.055%, 0.06%, 0.065%, 0.07%, 0.075%, 0.08%, 0.085%, 0.09%, 0.095%, 0.1%, 0.105%, or 0.11% (w/v) or greater.

68. The method of any one of claims 66-67, wherein the concentration of the surfactant is between 0.05% and 0.2%, or between 0.06% and 0.18%, or between 0.07% and 0.16%, or between 0.07% and 0.15%, or between 0.08% and 0.125%, or between 0.09% and 0.12%.

69. The method of any one of claims 66-68, wherein the crowding agent comprises one or more of polyethylene glycol (PEG), polyvinylpyrrolidone (PVP), dextran, bovine serum albumin (BSA), or an epichlorohydrin and sucrose copolymer (Ficoll).

70. The method of claim 69, wherein the crowding agent comprises PEG and the PEG has an average molecular weight of about 2,000 to about 14,000, or about 4,000 to about 12,000, or about 6,000 to about 10,000.

71. The method of claim 69 or 70, wherein the crowding agent comprises PVP with a molecular weight of about 4,000 to about 14,000, or about 6,000 to about 12,000, or about 8,000 to about 11,000.

72. The method of any one of claims 66-71, wherein the concentration of the crowding agent is between 1 mM and 10 mM.

73. The method of claim 72, wherein the concentration of the crowding agent is between 1 mM and 5 mM.

74. The method of any one of claims 66-73, wherein the surfactant is a nonionic surfactant.

75. The method of claim 74, wherein the surfactant comprises one or more of polysorbate 20 (TWEEN 20), polysorbate 80 (TWEEN 80), 2-[4-(2,4,4-trimethylpentan-2-yl)phenoxy]ethanol (Triton X-100), an alcohol ethoxylate (e.g., Ecosurf EH-9, TERGITOL 15-S-9), nonyl phenoxypolyethoxylethanol (NP-40), 3-((3-cholamidopropyl) dimethylammonio)-1-propanesulfonate (CHAPS), 3-[(3-cholamidopropyl)dimethylammonio]-2-hydroxy-1-propanesulfonate (CHAPSO), polyoxyethylene (23) lauryl ether (Brij-35), polyoxyethylene (20) cetyl ether (Brij-58), polyoxyethylene (20) stearyl ether (Brij-78), or polyoxyethylene (20) oleyl ether (Brij-98).

76. The method of any one of claims 66-75, wherein the surfactant comprises a cationic or zwitterionic surfactant.

77. The method of claim 76, wherein the surfactant comprises one or more of benzalkonium chloride (BZK), didodecyldimethylammonium bromide (DDAB), lauryldimethylamine oxide (LDAO), an amine betaine (e.g., N,N-Dimethyl-N-dodecylglycine betaine (EMPIGEN BB)), or an ammonium sulfonate (e.g., n-Tetradecyl-N,N-dimethyl-3-ammonio-1-propanesulfonate (ZWITTERGENT 3-14)).

78. The method of any one of claims 66-77, wherein said DNA polymerase is a thermostable DNA polymerase.

79. The method of claim 78, wherein said thermostable DNA polymerase Taq DNA polymerase or a mutant, variant, or derivative thereof.

80. The method of any one of claims 66-79, further comprising one or more nucleotides (dNTPs).

81. The method of claim 80, wherein said nucleotides are selected from the group consisting of dTTP, dATP, dCTP, dGTP, 7-deaza-dGTP or dUTP.

82. The method of claim 80 or 81, wherein the concentration of each of said nucleotides is about 0.5 mM to 5 mM.

83. The method of any one of claims 66-82, further comprising a carrier.

84. The method of claim 83, wherein the carrier is glycerol.

85. The method of claim 84, wherein the concentration of said glycerol is between 5%-50%.

86. The method of any one of claims 66-85, further comprising a reference control.

87. The method of claim 86, wherein the reference control is a dye.

88. The method of claim 87, wherein the reference control is a ROX dye.

89. The method of any one of claims 66-88, wherein said composition is a concentrated stock solution.

90. The method of claim 89, wherein said concentrated stock solution is a 2× to 5× stock solution.

91. The method of any one of claims 66-90, wherein the viscosity of the composition ranges between about 1 cP to about 20 cP, or about 1 cP to about 15 cP, or about 1 cP to about 10 cP, or about 3 cP to 8 cP.

92. A method for amplifying a nucleic acid molecule, said method comprising:
(a) mixing a composition with a nucleic acid sample, the composition comprising:
at least one DNA polymerase,
at least one reverse transcriptase,
at least one crowding agent, and
at least one surfactant at a concentration of at least 0.05% (w/v); and
(b) incubating the mixture under conditions sufficient to amplify a nucleic acid molecule complementary to all or a portion of said nucleic acid template.

93. The method of claim 92, wherein the concentration of the surfactant is at least 0.055%, 0.06%, 0.065%, 0.07%, 0.075%, 0.08%, 0.085%, 0.09%, 0.095%, 0.1%, 0.105%, or 0.11% (w/v) or greater.

94. The method of any one of claims 92-93, wherein the concentration of the surfactant is between 0.05% and 0.2%, or between 0.06% and 0.18%, or between 0.07% and 0.16%, or between 0.07% and 0.15%, or between 0.08% and 0.125%, or between 0.09% and 0.12%.

95. The method of any one of claims 92-94, wherein the crowding agent comprises one or more of polyethylene glycol (PEG), polyvinylpyrrolidone (PVP), dextran, bovine serum albumin (BSA), or an epichlorohydrin and sucrose copolymer (Ficoll).

96. The method of claim 95, wherein the crowding agent comprises PEG and the PEG has an average molecular weight of about 2,000 to about 14,000, or about 4,000 to about 12,000, or about 6,000 to about 10,000.

97. The method of claim 95 or 96, wherein the crowding agent comprises PVP with a molecular weight of about 4,000 to about 14,000, or about 6,000 to about 12,000, or about 8,000 to about 11,000.

98. The method of any one of claims 92-97, wherein the concentration of the crowding agent is between 1 mM and 10 mM.

99. The method of claim 98, wherein the concentration of the crowding agent is between 1 mM and 5 mM.

100. The method of any one of claims 92-99, wherein the surfactant is a nonionic surfactant.

101. The method of claim 100, wherein the surfactant comprises one or more of polysorbate 20 (TWEEN 20), polysorbate 80 (TWEEN 80), 2-[4-(2,4,4-trimethylpentan-2-yl)phenoxy]ethanol (Triton X-100), an alcohol ethoxylate (e.g., Ecosurf EH-9, TERGITOL 15-S-9), nonyl phenoxypolyethoxylethanol (NP-40), 3-((3-cholamidopropyl) dimethylammonio)-1-propanesulfonate (CHAPS), 3-[(3-cholamidopropyl)dimethylammonio]-2-hydroxy-1-propanesulfonate (CHAPSO), polyoxyethylene (23) lauryl ether (Brij-35), polyoxyethylene (20) cetyl ether (Brij-58), polyoxyethylene (20) stearyl ether (Brij-78), or polyoxyethylene (20) oleyl ether (Brij-98).

102. The method of any one of claims 92-101, wherein the surfactant comprises a cationic or zwitterionic surfactant.

103. The method of claim 102, wherein the surfactant comprises one or more of benzalkonium chloride (BZK), didodecyldimethylammonium bromide (DDAB), lauryldimethylamine oxide (LDAO), an amine betaine (e.g., N,N-Dimethyl-N-dodecylglycine betaine (EMPIGEN BB)), or an ammonium sulfonate (e.g., n-Tetradecyl-N,N-dimethyl-3-ammonio-1-propanesulfonate (ZWITTERGENT 3-14)).

104. The method of any one of claims 92-103, wherein said DNA polymerase is a thermostable DNA polymerase.

105. The method of claim 104, wherein said thermostable DNA polymerase Taq DNA polymerase or a mutant, variant, or derivative thereof.

106. The method of any one of claims 92-105, further comprising one or more nucleotides (dNTPs).

107. The method of claim 106, wherein said nucleotides are selected from the group consisting of dTTP, dATP, dCTP, dGTP, 7-deaza-dGTP, or dUTP.

108. The method of claim 106 or 107, wherein the concentration of each of said nucleotides is about 0.5 mM to 5 mM.

109. The method of any one of claims 92-108, further comprising a carrier.

110. The method of claim 109, wherein the carrier is glycerol.

111. The method of claim 110, wherein the concentration of said glycerol is between 5%-50%.

112. The method of any one of claims 92-111, further comprising a reference control.

113. The method of claim 112, wherein the reference control is a dye.

114. The method of claim 113, wherein the reference control is a ROX dye.

115. The method of any one of claims 92-114, wherein said composition is a concentrated stock solution.

116. The method of any one of claims 92-115, where said concentrated stock solution is a 2× to 5× stock solution.

117. The method of any one of claims 92-116, wherein the viscosity of the composition ranges between about 1 cP to about 20 cP, or about 1 cP to about 15 cP, or about 1 cP to about 10 cP, or about 3 cP to 8 cP.

118. A kit, comprising in a single container a composition as in any one of claims 1-30.

119. The kit of claim 118, wherein the composition further comprises a reverse transcriptase.
